(19) United States
(12) Patent Application Publication
(10) Pub. No.: US 2026/0048189 A1
(43) Pub. Date: Feb. 19, 2026

(54) EXTRACORPOREAL BLOOD TREATMENT FOR ORGAN SUPPORT

(71) Applicants: Vantive US Healthcare LLC, Deerfield, IL (US); Vantive Health GmbH, Glattpark (Opfikon) (CH)

(72) Inventors: Timothy Nadolski, Maple Grove, MN (US); Klaus Obergfell, Minneapolis, MN (US); William West, Maple Grove, MN (US); Timothy Byland, Plymouth, MN (US); Jason Knox, Bellambi NSW (AU)

(21) Appl. No.: 19/129,797

(22) PCT Filed: Nov. 1, 2023
(86) PCT No.: PCT/EP2023/080478

§ 371 Date: May 14, 2025

Related U.S. Application Data
(60) Provisional application No. 63/426,575, filed on Nov. 18, 2022.

(51) Int. Cl. A61M 1/36; A61M 1/16

(52) U.S. Cl. CPC A61M 1/3603; A61M 1/16; A61M 1/3607; A61M 1/3626; A61M 1/3627; A61M 1/3653; A61M 1/367; A61M 2202/0021; A61M 2202/0413; A61M 2205/3327; A61M 2205/3334; A61M 2205/3344; A61M 2205/50; A61M 2205/75

(57) ABSTRACT

Extracorporeal blood treatment apparatus, methods, and systems to perform an extracorporeal blood treatment using an organic filter operable to perform the functions of an organ are described herein. The extracorporeal blood treatment apparatus includes a treatment set, a high flow pump, a first low flow pump, and a second low flow pump, and a computing apparatus operatively coupled to the extracorporeal blood treatment apparatus. The computing apparatus may be configured to perform an extracorporeal blood treatment using the extracorporeal blood treatment apparatus and the organic filter.

The disclosure herein relates to extracorporeal blood treatment using an extracorporeal blood treatment apparatus. More particularly, the disclosure relates to devices, apparatus, methods, systems, and treatment sets for extracorporeal blood treatment for organ support.

Extracorporeal blood treatment may refer to taking blood from a patient, treating the blood outside the patient, and returning the treated blood to the patient. Extracorporeal blood treatment is typically used to extract undesirable matter or molecules from the patient's blood, and/or to add beneficial matter or molecules to the blood. Extracorporeal blood treatment may be used with patients incapable of effectively eliminating matter from their blood, for example, in the case of a patient who is suffering from temporary or permanent liver failure. These and other patients may, for instance, undergo extracorporeal blood treatment to add to or to eliminate matter from their blood, to maintain an acid-base balance or to eliminate excess body fluids or toxins.

In a variety of extracorporeal blood treatments, one or more fluids, or liquids, may be supplied to the extracorporeal blood treatment apparatus for use during the treatments and one or more fluids may be collected as a part of the treatments. Various kit and tubing configurations, and various medical devices, may be used depending on the extracorporeal blood treatment.

### SUMMARY

The present disclosure describes one or more illustrative embodiments of devices, apparatus, systems and methods that provide extracorporeal blood treatment for organ support for patients. For example, an extracorporeal blood treatment apparatus or an extracorporeal blood treatment system may be utilized to perform an extracorporeal blood treatment on a patient using an organic filter operable to perform the functions of a human liver organ. In such cases, blood may be drawn from the patient and passed through the organic filter before being returned to the patient. The organic filter may be, for example, a liver transplant organ or synthetic liver organ which functions as an organic liver as discussed herein, or a bioengineered organ, artificial organ, bio-hybrid organ, etc. In alternative embodiments, the apparatus, devices, systems, and methods may instead be used for kidney support and use an organic filter operable to perform the functions of a human kidney organ. In alternative embodiments, the apparatus, devices, systems, and methods may instead be used for lung support and use an organic filter operable to perform the functions of a human lung organ. In alternative embodiments, unfiltered blood is drawn not from a patient's vasculature, but from a storage container (e.g., a bag of blood).

The apparatus or system may also include a computing apparatus which can control the extracorporeal blood treatment for organ support. The computing apparatus may control flow rates of various pumps of the extracorporeal blood treatment apparatus in order to achieve or maintain pressure at certain locations in a treatment set or to achieve or maintain weight of various components of the extracorporeal blood treatment apparatus, and may control various flow rates of various pumps based on other variables such as desired flow rate or amount of a substance or toxin in the blood. The computing apparatus may control and adjust flow rates of various pumps in order to control the pressures or weights as described herein.

One illustrative extracorporeal blood treatment system may include an apparatus to perform an extracorporeal blood treatment. The apparatus may perform the treatment using an organic filter. The organic filter may be operable to perform functions of a human organ. The apparatus may include a treatment set. The treatment set may include tubing. The treatment set may be operatively couplable to the patient. The treatment set may be configured to move blood from the patient to the organic filter. The treatment set may return the filtered blood to the patient. The treatment set may include a high flow pump. The high flow pump may be operatively couplable to the treatment set to move blood through the treatment set from a buffer reservoir towards the organic filter. The treatment set may include a first low flow pump. The first low flow pump may be operatively couplable to the treatment set to move blood through the treatment set from the patient towards the organic filter. The treatment set may include a second low flow pump. The second low flow pump may be operatively couplable to the treatment set to move blood through the treatment set from the organic filter towards the patient. The apparatus may include a computing apparatus. The computing apparatus may be operatively coupled to the pumps. The computing apparatus may be configured to perform an extracorporeal blood treatment by controlling the pumps.

One illustrative treatment set may include an access line. The access line may be operatively couplable to a patient. The access line may be configured to receive blood from the patient. The treatment set may include a buffer reservoir line. The buffer reservoir line may be operatively couplable to a buffer reservoir. The buffer reservoir line may be configured to receive blood from the buffer reservoir. The treatment set may include an organic filter inlet line. The organic filter inlet line may be operatively couplable to the access line, the buffer reservoir line, and an organic filter. The organic filter inlet line may be configured to move blood from the access line and the buffer reservoir line to the organic filter. The treatment set may include an organic filter outlet line. The organic filter outlet line may be operatively couplable to the organic filter and the buffer reservoir. The organic filter outlet line may be configured to move blood from the organic filter to the buffer reservoir. The treatment set may include a return line. The return line may be operatively couplable to the buffer reservoir and the patient. The return line may be configured to move blood from the buffer reservoir to the patient. The treatment set may include a runoff line. The runoff line may be operatively couplable to the buffer reservoir. The runoff line may be configured to move fluid runoff from the organic filter to the buffer reservoir.

One illustrative method may include monitoring at least one of an access pressure, an organic filter pressure, a return pressure, and a buffer reservoir weight of an extracorporeal blood treatment device to perform an extracorporeal blood treatment using an organic filter operable to perform functions of a human organ. Monitoring of the access pressure may be performed using an access pressure sensor. Monitoring of the organic filter pressure may be performed using an organic filter pressure sensor. Monitoring of the return pressure may be performed using a return pressure sensor. Monitoring of the buffer reservoir weight may be performed using a reservoir scale. The method may further include adjusting a pump speed of at least one of a first low flow pump, a second low flow pump, and a high flow pump based on the monitored at least one of the access pressure, the organic filter pressure, the return pressure, and the buffer reservoir weight.

One illustrative computer readable medium is configured to monitor at least one of an access pressure, an organic filter pressure, a return pressure, and a buffer reservoir weight of an extracorporeal blood treatment device and an organic filter operable to perform functions of a human organ. Monitoring of the access pressure may be performed using an access pressure sensor. Monitoring of the organic filter pressure may be performed using an organic filter pressure sensor. Monitoring of the return pressure may be performed using a return pressure sensor. Monitoring of the buffer reservoir weight may be performed using a reservoir scale. The computer readable medium may be further configured to adjust a pump speed of at least one of a first low flow pump, a second low flow pump, and a high flow pump based on the monitored at least one of the access pressure, the organic filter pressure, the return pressure, and the buffer reservoir weight.

One illustrative device may include a device to perform an extracorporeal blood treatment using an organic filter operable to perform functions of a human organ. The device may include a computing apparatus operatively coupled to at least one pump. The computing apparatus may be configured to perform an extracorporeal blood treatment by controlling the at least one pump.

One illustrative extracorporeal blood treatment apparatus may include a treatment set. The treatment set may include tubing. The treatment set may be operatively couplable to a patient. The treatment set may be configured to move blood from the patient to an organic filter. The organic filter may be operable to perform functions of a human organ. The treatment set may be configured to return the filtered blood to the patient. The treatment set may include an access line. The access line may be operatively couplable to a patient and configured to receive blood from the patient. The treatment set may include an organic filter inlet line. The organic filter inlet line may be operatively couplable to the access line and to the organic filter. The organic filter inlet line may be configured to move blood from the access line to the organic filter. The treatment set may include a return line. The return line may be operatively couplable to the patient and configured to move blood back to the patient. The treatment set may include a high flow pump outlet line. The high flow pump outlet line may be operatively couplable to at least one of the access line and the organic filter inlet line. The high flow pump outlet line may be configured to move blood from the organic filter to at least one of the access line and the organic filter inlet line. The apparatus may further include a first low flow pump. The first low flow pump may be operatively couplable to the access line. The first low flow pump may be configured to move blood through the treatment set from the patient towards the organic filter. The apparatus may include a second low flow pump. The second low flow pump may be operatively couplable to the return line. The second low flow pump may be configured to move blood through the treatment set from the organic filter towards the patient. The apparatus may include a high flow pump. The high flow pump may be operatively couplable to the high flow pump outlet line. The high flow pump may be configured to move blood from the organic filter to at least one of the access line and the organic filter inlet line. The high flow pump may be configured to move blood to the organic filter. The apparatus may include a computing apparatus. The computing apparatus may be operatively couplable to the pumps. The computing apparatus may be configured to control the first low flow pump to move blood through the treatment set from the patient towards the organic filter. The computing apparatus may be configured to control the second low flow pump to move blood through the treatment set from the organic filter towards the patient. The computing apparatus may be configured to control the high flow pump to move blood from the organic filter to either the access line or the organic filter inlet line. The computing apparatus may be configured to move blood to the organic filter.

The illustrative apparatus, devices, methods, and systems may be described as providing and facilitating a method for accessing blood from the patient, supporting the determined pressures and blood flows to keep the organic filter viable, a way to account for blood losses from the organic filter, and return the blood back to the patient. Specifically, the organic filters may need specific temperatures, blood flow, pressures and oxygen to mimic their normal operating environment, and the illustrative apparatus or system may be set up to be able to provide these parameters many of these outside of the human body by, for example, using a combination of pumps to meet the blood flow rates, using the embedded pressure sensors to monitor and control the pressures to the organic filter, by using the embedded pressure sensors to monitor and control the blood access and return to the patient, and using the embedded scales to monitor and control the blood loss in the system.

The illustrative devices, apparatus, methods, and systems may be generally described as include two major elements that is a computing apparatus, or control unit, and a disposable treatment set that is connected to the patient via a central venous access. Blood may be withdrawn from the patient via an access line of the treatment set connected to the central venous access and then circulated through an oxygenator, an organic filter located in an organ chamber and via a blood bag before the cleaned blood is returned to the patient via the return line connected to the central venous access. In alternative embodiments, unfiltered blood is drawn not from a patient's vasculature, but from a storage container (e.g., a bag of blood). The extracorporeal circuit defined by the treatment set may also have a recirculation loop within the circuit that will circulate the blood through an oxygenator and the organic filter at a higher blood flow rate than the blood flow rate used when the blood is withdrawn from the patient. The oxygenator may be used to oxygenate the blood withdrawn from the patient's venous circulation before it is circulated through the organic filter. The organic filter may clean blood from the toxins via adsorption before it is returned to the patient.

The computing apparatus, or control unit, may be used for pumping the blood through the extracorporeal circuit using two of the systems'peristaltic low-flow pumps. The blood flow rates of the two low-flow pumps may be possible to set between 0-166 ml/min. The flow rates of the individual pump may be manually adjusted by the operator or may be automatically adjusted by the computing apparatus to keep the blood volume in the blood bag within a predefined range. The computing apparatus may also be used for pumping the blood through the recirculation loop within the extracorporeal circuit using the systems'peristaltic high-flow pump. The recirculation blood flow may be possible to set between 0-450 ml/min.

The computing apparatus may be further used for monitoring that the blood volume in the blood bag of the disposable set remains stable within certain limits, utilizing one of the scales of the system. The blood bag may be hanging on the scale and the weight of the bag may be monitored against a tare weight that is determined at the start of therapy. The purpose of the blood bag may be to be a blood reservoir for the recirculation circuit and to collect any blood from the bottom of the organ chamber (e.g., fluid wept from the organic filter).

The computing apparatus may be further used for monitoring of the access (36a), organic filter (36b) and return (36c) pressure in the extracorporeal circuit, the liquid level in the deaeration chamber (40), as well as monitoring for air bubbles in the return line using the ultrasonic air detector (42) and preventing detected air bubbles from reaching the patient using the return line clamp.

The disposable treatment set may have several sample ports placed on strategic places throughout the extracorporeal circuit to enable blood sampling (38a-d).

The illustrative devices, apparatus, methods, and systems may be described as having a therapy mode that supports the external liver assist product (ELAP) therapy and the disposable treatment set. In the ELAP therapy, anticoagulation of the extracorporeal circuit of the disposable treatment set will be performed by infusing anticoagulant solution using a syringe/infusion pump. Further, the system may provide automatic loading of the disposable treatment set using the automatic loading functionality of the system. Also, the disposable treatment set may be identifiable via barcodes, on the front and/or the back of the disposable treatment set and read by the systems barcode reader.

The illustrative devices, apparatus, methods, and systems may provide a manual or automatic priming procedure for the disposable treatment set where the operator may need to manually start and stop the three blood pumps at a certain flow rates in order to prime the extracorporeal circuit of the disposable treatment set. The used priming solution may be collected in a separate collection bag, that may not be monitored.

In the ELAP therapy, the illustrative devices, apparatus, methods, and systems may request for the medical prescription via the following parameters: Blood Pump Access (BP-Access) flow rate; Blood Pump Return (BP-Return) flow rate; and Blood Pump Organ (BP-Organ) flow rate. Further, the illustrative apparatus or system may allow for the following blood flow rates for the above blood pumps: a blood flow rate up to 166 ml/min for the Blood Pump Access (peristaltic low-flow pump); a blood flow rate up to 166 ml/min for the Blood Pump Return (peristaltic low-flow pump); and a blood flow rate up to 450 ml/min for the Blood Pump Organ (peristaltic high-flow pump).

The blood flow rate accuracy for all the above blood pumps may be less than ±10% at the highest user settable blood flow, with a fluid temperature of 37° C., at an access pressure of −200 mmHg. Access pressure is defined as the pressure measured in the blood withdrawal line of the extracorporeal circuit at the position of the access pod.

The illustrative devices, apparatus, methods, and systems may monitor the pressures in the extracorporeal circuit of the disposable treatment set utilizing the systems'pressure sensors. The following pressures may be monitored: access pressure, before the first low-flow blood pump (Pump 1), using the access pressure sensor (pod based); organ pressure, before the organic filter in the organ chamber, using the organic filter pressure sensor (pod based); and return pressure, at the top of the deaeration chamber after the second low-flow blood pump (Pump 2), using the return pressure sensor. The pressure operating ranges and accuracies of the illustrative apparatus or system may be as follows: measure the access pressure in the range of −250 to +450 mmHg with an accuracy better than ±15 mmHg; measure the return pressure in the range of −50 to +350 mmHg with an accuracy better than ±5 mmHg; and measure the organ pressure in the range of −50 to +450 mmHg with an accuracy better than ±50 mmHg.

The above summary of the present disclosure is not intended to describe each embodiment or every implementation thereof. Advantages, together with a more complete understanding of the present disclosure, will become apparent and appreciated by referring to the following detailed description and claims taken in conjunction with the accompanying drawings.

### BRIEF DESCRIPTION OF THE DRAWING

FIG. 1 is a block diagram of an illustrative extracorporeal blood treatment system including input apparatus, display apparatus, and extracorporeal blood treatment apparatus that may utilize the systems and treatment sets described herein.

FIG. 2 is a depiction of an illustrative extracorporeal blood treatment apparatus that includes a computing apparatus as described herein.

FIG. 3 is a diagram of an illustrative fluid circuit that may be used by the extracorporeal treatment systems and apparatuses as described herein to perform an extracorporeal blood treatment on a patient using an organic filter operable to perform the functions of a human organ.

FIG. 4 is a depiction of an illustrative treatment set.

FIG. 5 is an illustrative method using the extracorporeal blood treatment apparatus or system as illustrated in FIGS. 1-4.

### DETAILED DESCRIPTION OF EXEMPLARY EMBODIMENTS

In the following detailed description of illustrative embodiments, reference is made to the accompanying figures of the drawing which form a part hereof, and in which are shown, by way of illustration, specific embodiments which may be practiced. It is to be understood that other embodiments may be utilized and structural changes may be made without departing from (e.g., still falling within) the scope of the disclosure presented hereby.

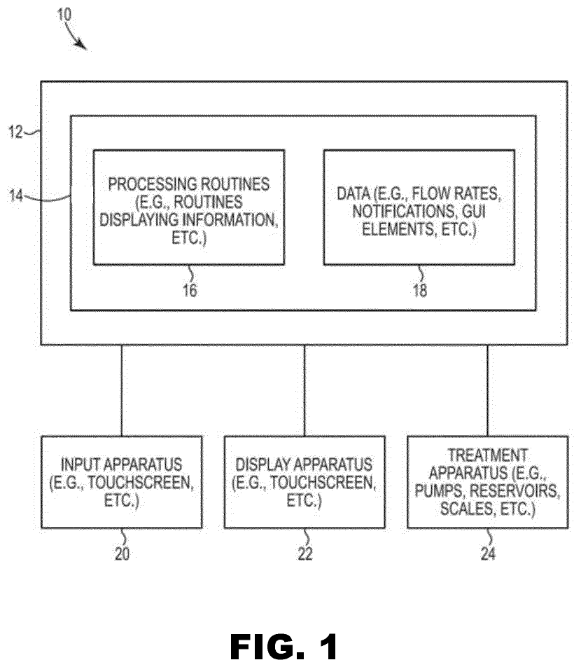

Illustrative devices, apparatus, methods, systems and treatment sets for extracorporeal blood treatment for organ support shall be described with reference to FIGS. 1-5. It will be apparent to one skilled in the art that elements or processes from one embodiment may be used in combination with elements or processes of the other embodiments, and that the possible embodiments of such systems and methods using combinations of features set forth herein is not limited to the specific embodiments shown in the Figures and/or described herein. Further, it will be recognized that the embodiments described herein may include many elements that are not necessarily shown to scale. Still further, it will be recognized that timing of the processes and the size and shape of various elements herein may be modified but still fall within the scope of the present disclosure, although certain timings, one or more shapes and/or sizes, or types of elements, may be advantageous over others.

The illustrative devices, apparatus, methods, and systems include an extracorporeal blood treatment apparatus including elements or components such as, e.g., pumps, tubing/treatment sets, reservoirs, etc., to perform an extracorporeal blood treatment on a patient using an organic filter operable to perform the functions of a human organ. In some cases, the organic filter may be a liver organ such as described herein. In at least one embodiment, the extracorporeal blood treatment apparatus can include, among other things, a treatment set, a high flow pump, a first low flow pump and a second low flow pump. The high flow pump may be configured to pump fluid or fluid in a tubing or treatment set at a flow rate that is higher than the first and second low flow pumps. In alternative embodiments, contrary to the nomenclature, the high flow pump may be configured to pump fluid at a flow rate that is the same or lower than the first and second low flow pumps. The nomenclature is not meant to be limiting in terms of absolute flow rate, but instead to differentiate between the pumps.

The organic filter may use a minimum volume and/or minimum flow rate of blood moving through it at any given moment to effectively function to perform the liver functionality. In other words, if a minimum volume and a minimum flow rate of blood are not maintained in the organic filter, the organic filter may not effectively perform the extracorporeal blood treatment.

Organs are challenging to reproduce synthetically, at least because the anatomical structure of organs is highly complex. In some instances, porcine organs may be used as the matrix for creating human organs, due in part to anatomical and vascular similarities between the two species. Porcine organs may be decellularized to remove porcine cells, leaving behind a scaffold extracellular matrix. This scaffold may then be recellularized, or populated with, functional living human cells. Such human cells may be isolated from donated human organs, for example. The recellularized organic liver may function as a normal human liver and may offer a lower risk of rejection when used with a human patient.

One example of an organic filter as described herein can be the MIROMATRIX™ perfusion decellularization and recellularization of a porcine organ. An example of decellularization and recellularization of organs and tissues is shown in U.S. Pat. Pub. No. 2022/0062349 A1, filed on Aug. 4, 2021, by Taylor et al. Illustrative methods of using blood to increase engraftment of human cells on a biocompatible scaffold is shown in U.S. Pat. No. 11,278,643 B2, filed on Sep. 6, 2017, by Ross et al. Illustrative methods of decellularization and recellularization of solid organs is shown in U.S. Pat. Pub. No. 2019/0343877 A1, filed on Jan. 11, 2019, by Ott et al. Illustrative methods of preparing a graft from a recellularized mammalian liver is shown in U.S. Pat. No. 11,452,797 B2, filed on Jan. 9, 2019, by Jeffrey Ross. Illustrative methods of using a perfusion decellularized organ for matched recellularization is shown in EP U.S. Pat. No. 2,588,592 B2, filed on Jun. 30, 2011, by Jeffrey Ross. Illustrative systems and apparatuses for initial preparation of an organ scaffold to form an artificial organ is shown in U.S. Pat. Pub. No. 2010/0093066 A1, filed on Aug. 25, 2009, by Taylor et al.

In alternative embodiments, the apparatus, devices, systems, and methods may instead be used for kidney support and use an organic filter operable to perform the functions of a kidney organ. In alternative embodiments, the apparatus, devices, systems, and methods may instead be used for lung support and use an organic filter operable to perform the functions of a lung organ.

To maintain the minimum volume and minimum flow rate for the organic filter, the illustrative apparatus and system utilize multiple pumps usable together and a buffer reservoir as will be described further herein. The pumps include a high flow pump and two low flow pumps. The outputs of the high flow pump and one of the low flow pumps are combined to provide the minimum flow rate for the organic filter. The buffer reservoir, which may be a hanging bag of blood, may be used to ensure there is enough blood in the system or treatment set to satisfy the minimum volume and/or flow rate of the organic filter. In other words, the pumps and the buffer reservoir as configured herein can ensure that there is enough blood to flow to exceed the minimum flow rate through and enough blood to exceed the minimum volume in the organic filter as used by the organic filter.

Additionally, the illustrative apparatus or system may be controllable to maintain or control pressure at certain locations in a tubing, or treatment, set, such as an access pressure proximate the patient where blood is withdrawn from the patient or storage container, a return pressure proximate the patient where filtered blood is returned to the patient, or an organic filter pressure proximate the organic filter. For example, the system may include a computing apparatus that may control the high flow pump, the first low flow pump, and the second low flow pump in order to control the pressures, as further discussed herein. Further, the computer apparatus may maintain or control weight of various components such as the buffer reservoir. Still further, the computing apparatus may control the high flow pump, the first low flow pump, and the second low flow pump in order to control the weight, as discussed herein. Still further, the computing apparatus may maintain or control desired flow rates of the pumps or maintain or control the amount of a substance or toxin in the blood.

The flow rate of a fluid in the system or treatment set may be adjusted. The flow rate of each fluid may be limited (e.g., upper and lower flow rate limits) based on multiple factors including but not limited to the other flow rates of the extracorporeal blood treatment system, pressure at different locations in the treatment set, weight of various components of the system, etc. When a selected flow rate is adjusted to a limit, or the limit has been reached, the exemplary systems may, for example, provide a notification to a user to adjust or modify the flow rate or to pause the extracorporeal blood treatment.

An illustrative extracorporeal blood treatment system 10 depicted in FIG. 1 may be used to execute, or perform, the exemplary methods and/or processes described herein. In at least one embodiment, the system 10 may be a machine for the extracorporeal treatment of blood. The system 10 could, for example, alternatively be a blood processing device or a blood component preparation device or other medical apparatus for fluid delivery/collection.

As shown, the exemplary extracorporeal blood treatment system 10 includes computing apparatus 12. The computing apparatus 12 may be configured to receive input from input apparatus 20 and transmit output to display apparatus 22. Further, the computing apparatus 12 may include data storage 14. Data storage 14 may allow for access to processing programs or routines 16 and one or more other types of data 18 that may be employed to carry out exemplary methods and/or processes (e.g., measuring pressures, synchronized pumps, weighing reservoirs, adjusting treatments, adjusting flow rates, calculating flow rates, determining flow rates dependent on other flow rates, running a treatment, notifying operator, or users, of problems, displaying status information, etc.) for use in performing extracorporeal blood treatments. For example, the computing apparatus 12 may be configured to display an exemplary graphical user interface displayed by the display apparatus 22 including a treatment set with one or more fluid lines, various pumps as described herein, flow rates or pressures within the treatment set, weight of various components, etc.

The computing apparatus 12 may be operatively coupled to the input apparatus 20 and the display apparatus 22 to, e.g., transmit data to and from each of the input apparatus 20 and the display apparatus 22. For example, the computing apparatus 12 may be electrically coupled to each of the input apparatus 20 and the display apparatus 22 using, e.g., analog electrical connections, digital electrical connections, wireless connections, bus-based connections, etc. For example, an operator may provide input to the input apparatus 20 to manipulate or modify one or more graphical depictions displayed on the display apparatus 22 to select and adjust one or more flow rates, pressures, weights, etc. during, before, or after any extracorporeal blood treatments.

Further, various devices and apparatus may be operatively coupled to the computing apparatus 12 to be used with the computing apparatus 12 to perform one or more extracorporeal procedures/treatments as well as the functionality, methods, and/or logic described herein. As shown, the system 10 may include input apparatus 20, display apparatus 22, and treatment apparatus 24 operatively coupled to the computing apparatus 12 (e.g., such that the computing apparatus 12 may be configured to use information, or data, from the apparatuses 20, 22, 24 and provide information, or data, to the apparatuses 20, 22, 24). The input apparatus 20 may include any apparatus capable of providing input to the computing apparatus 12 to perform the functionality, methods, and/or logic described herein.

For example, the input apparatus 20 may include a touchscreen (e.g., capacitive touchscreen, a resistive touchscreen, a multi-touch touchscreen, etc.), a mouse, a keyboard, a trackball, etc. A touchscreen may overlay the display apparatus 22 such that, e.g., an operator may use the touchscreen to interact (e.g., by touch) with a graphical user interface displayed on the display apparatus. The input apparatus 20 may allow an operator to interact with a graphical user interface including a treatment set with one or more fluid lines, various pumps as described herein, flow rates or pressures within the treatment set, weight of various components, etc., when used in conjunction with the display apparatus 22 (e.g., displaying the graphical user interface).

The display apparatus 22 may include any apparatus capable of displaying information to an operator, such as a graphical user interface, etc., to perform the functionality, methods, and/or logic described herein. For example, the display apparatus 22 may include a liquid crystal display, an organic light-emitting diode screen, a touchscreen, a cathode ray tube display, etc. As described further herein, the graphical user interface displayed by the display apparatus 22 may include multiple items related to the extracorporeal blood treatment such as, e.g., a treatment set with one or more fluid lines, various pumps as described herein, flow rates or pressures within the treatment set, weight of various components, etc. Further, each of these components may be used, or interacted with, by a user to change, or modify, one or more parameters associated with the fluid within the treatment set, such as flow rate, pressure, concentration, etc.

The processing programs or routines 16 may include programs or routines for performing computational mathematics, matrix mathematics, standardization algorithms, comparison algorithms, or any other processing required to implement one or more exemplary methods and/or processes described herein. Data 18 may include, for example, organic filters limits, fluid data, flow rates, fluid volumes, notifications, pressures, blood flow, fluid removal rates, target blood temperatures, graphics (e.g., graphical elements, icons, buttons, windows, dialogs, pull-down menus, graphic areas, graphic regions, 3D graphics, etc.), graphical user interfaces, results from one or more processing programs or routines employed according to the disclosure herein, or any other data that may be necessary for carrying out the one and/or more processes or methods described herein.

In one or more embodiments, the system 10 may be implemented using one or more computer programs executed on programmable computers, such as computers that include, for example, processing capabilities, data storage (e.g., volatile or non-volatile memory and/or storage elements), input devices, and output devices. Program code and/or logic described herein may be applied to input data to perform functionality described herein and generate desired output information. The output information may be applied as input to one or more other devices and/or methods as described herein or as would be applied in a known fashion.

The program used to implement the methods and/or processes described herein may be provided using any programmable language, e.g., a high level procedural and/or object orientated programming language that is suitable for communicating with a computer system. Any such programs may, for example, be stored on any suitable device, e.g., a storage media, that is readable by a general or special purpose program running on a computer system (e.g., including processing apparatus) for configuring and operating the computer system when the suitable device is read for performing the procedures described herein. In other words, at least in one embodiment, the system 10 may be implemented using a computer readable storage medium, configured with a computer program, where the storage medium so configured causes the computer to operate in a specific and predefined manner to perform functions described herein. Further, in at least one embodiment, the system 10 may be described as being implemented by logic (e.g., object code) encoded in one or more non-transitory media that includes code for execution and, when executed by a processor, is operable to perform operations such as the methods, processes, and/or functionality described herein.

Likewise, the system 10 may be configured at a remote site (e.g., an application server) that allows access by one or more operator, or users, via a remote computer apparatus (e.g., via a web browser), and allows an operator to employ the functionality according to the present disclosure (e.g., an operator accesses a graphical user interface associated with one or more programs to process data).

The computing apparatus 12 may be, for example, any fixed or mobile computer system (e.g., a controller, a microcontroller, a personal computer, mini computer, etc.). The exact configuration of the computing apparatus 12 is not limiting, and essentially any device capable of providing suitable computing capabilities and control capabilities (e.g., graphics processing, control of extracorporeal blood treatment apparatus, etc.) may be used.

As described herein, a digital file may be any medium (e.g., volatile or non-volatile memory, a CD-ROM, a punch card, magnetic recordable tape, etc.) containing digital bits (e.g., encoded in binary, trinary, etc.) that may be readable and/or writeable by computing apparatus 12 described herein. Also, as described herein, a file in user-readable format may be any representation of data (e.g., ASCII text, binary numbers, hexadecimal numbers, decimal numbers, graphically, etc.) presentable on any medium (e.g., paper, a display, etc.) readable and/or understandable by a user.

In view of the above, it will be readily apparent that the functionality as described in one or more embodiments according to the present disclosure may be implemented in any manner as would be known to one skilled in the art. As such, the computer language, the computer system, or any other software/hardware which is to be used to implement the processes described herein shall not be limiting on the scope of the systems, processes or programs (e.g., the functionality provided by such systems, processes or programs) described herein.

The methods and/or logic described in this disclosure, including those attributed to the systems, or various constituent components, may be implemented, at least in part, in hardware, software, firmware, or any combination thereof. For example, various aspects of the techniques may be implemented within one or more processors, including one or more microprocessors, DSPs, ASICs, FPGAS, or any other equivalent integrated or discrete logic circuitry, as well as any combinations of such components, or other devices. The term “processor” or “processing circuitry” may generally refer to any of the foregoing logic circuitry, alone or in combination with other logic circuitry, or any other equivalent circuitry.

Such hardware, software, and/or firmware may be implemented within the same device or within separate devices to support the various operations and functions described in this disclosure. In addition, any of the described components may be implemented together or separately as discrete but interoperable logic devices. Depiction of different features, e.g., using block diagrams, etc., is intended to highlight different functional aspects and does not necessarily imply that such features must be realized by separate hardware or software components. Rather, functionality may be performed by separate hardware or software components, or integrated within common or separate hardware or software components.

When implemented in software, the functionality ascribed to the systems, devices and methods described in this disclosure may be embodied as instructions and/or logic on a computer-readable medium such as RAM, ROM, NVRAM, EEPROM, FLASH memory, magnetic data storage media, optical data storage media, or the like. The instructions and/or logic may be executed by one or more processors to support one or more aspects of the functionality described in this disclosure.

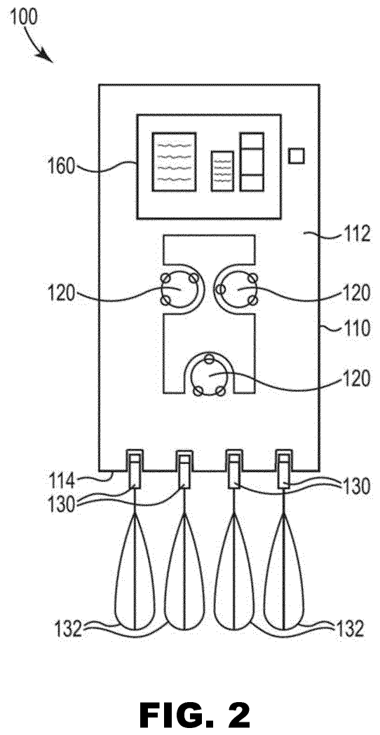

The treatment apparatus 24 may include any elements or components used by an exemplary extracorporeal blood treatment system capable of performing extracorporeal blood treatments, such as, e.g., pumps, reservoirs, scales, tubing, or treatment, sets, filters, pressure sensors, etc. For example, the treatment apparatus 24 may include one or more elements, or components, of the extracorporeal blood treatment system 100 described herein with reference to FIG. 2.

The illustrative apparatus, systems, and methods performed, or used, by such systems, described herein may be generally referred to as organ support systems. The systems, apparatus, and devices may be able to, for example, perform dialysis or other procedures or methods. The general term “dialysis” as used herein includes hemodialysis, hemofiltration, hemodiafiltration, hemoperfusion, liver dialysis, and therapeutic plasma exchange (TPE), among other similar treatment procedures. In dialysis generally, blood is taken out of the body and exposed to a treatment device to separate substances therefrom and/or to add substances thereto, and is then returned to the body. Although extracorporeal blood treatment systems capable of performing general dialysis (as defined above, including TPE) shall be described herein with reference to the illustrative extracorporeal blood treatment system of FIG. 2, other systems such as those for infusion of drugs, performance of continuous renal replacement therapy (CRRT), extracorporeal membrane oxygenation (ECMO), hemoperfusion, liver dialysis, apheresis, TPE, etc. may benefit from the systems, methods, and apparatus described herein and the present disclosure is not limited to any particular fluid processing system.

Referring to FIG. 2, one illustrative embodiment of an extracorporeal blood treatment system, or apparatus, 100 is depicted. The system 100 includes a housing 110 having a front face 112. The system further includes one or more pumps 120 used to move liquids through the apparatus as part of a treatment process. Although the pumps 120 are depicted in the form of peristaltic pumps, the pumps used in the extracorporeal blood treatment system described herein may be provided in a variety of alternative forms, e.g., piston pumps, pumps for use with syringes, diaphragm pumps, etc.

The extracorporeal blood treatment system 100 also includes, in one or more embodiments, a display 160 used to convey information to an operator. The display 160 may also serve as an input device if, e.g., the display 160 is in the form of a touchscreen. Also, although the display 160 is depicted as being located in the housing 110, in one or more alternate embodiments, the display 160 may be separate from the housing 110 of the extracorporeal blood treatment system 100. For example, the display 160 may be movably (e.g., swivel, tilt, etc.) attached, or coupled, to the housing 110.

The extracorporeal blood treatment system 100 may also include reservoir scales 130, each of which is configured to hold and weigh a reservoir 132. The reservoir scales 130 are positioned below a bottom end 114 of the housing 110, at least in part because the reservoirs 132 are typically attached to and hang from the reservoir scales 130. Although the depicted embodiment of extracorporeal blood treatment system 100 includes four reservoir scales 130 and associated reservoirs 132, alternative embodiments of an extracorporeal blood treatment apparatus as described herein may include one or more reservoir scales 130 and associated reservoirs 132 such as, e.g., as few as one reservoirs scale 130 and associated reservoir 132, four or more reservoirs scales 130 and associated reservoirs 132, etc.

In the embodiment shown, the reservoirs 132 may be in the form of, e.g., flexible polymeric bags configured to hold liquids. Reservoirs 132, however, used in connection with the exemplary extracorporeal blood treatment systems described herein may take any suitable form in which liquids can be stored and weighed by any scale or weighing apparatus (e.g., such as reservoir scales 130), e.g., bottles, tanks, cartons, syringes, jugs, etc.

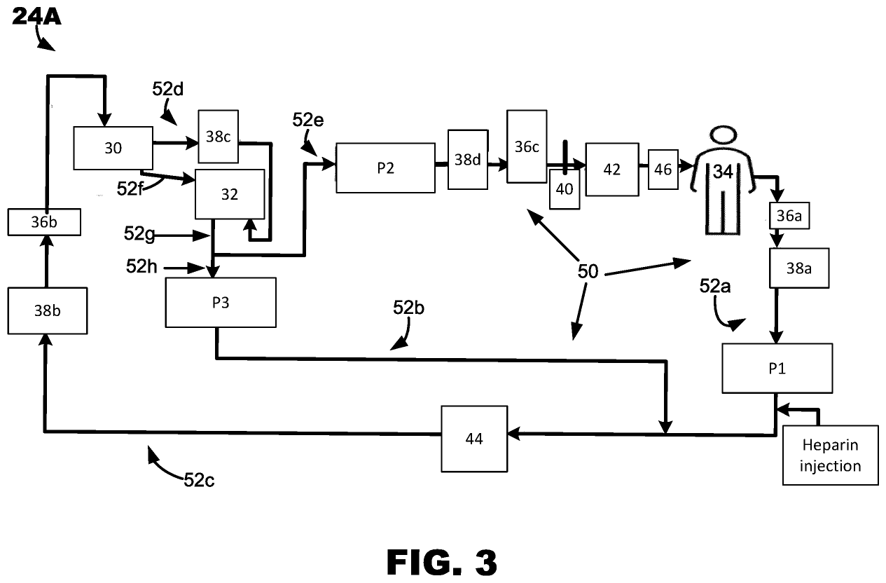

In the illustrative embodiment to perform an extracorporeal blood treatment as depicted diagrammatically in FIG. 3, the system may only utilize a single reservoir that is a buffer reservoir configured to maintain a buffer of blood to satisfy the flow rate and volume minimums used by the organic filter to maintain its functionality.

As shown in FIG. 1 and as related to FIG. 2, the treatment apparatus 24 may be operatively coupled, or connected, to the computing apparatus 12. Among the treatment apparatus 24 operably coupled to the computing apparatus 12 are the pumps 120 and reservoir scales 130 as shown in FIG. 2. Each of the pumps 120 and reservoirs 132 may have a flow rate associated therewith.

The computing apparatus 12 may, in one or more embodiments, be configured to receive a weight signal from each reservoir scale 130, with the weight signal from each reservoir scale 130 being indicative of the weight of a reservoir 132 attached to the reservoir scale 130. The computing apparatus 12 may further be configured to make a determination that the reservoir 132 attached to the reservoir scale 130 from which the weight signal has been received has risen or decreased passed a selected weight limit at least partially based on the weight signal received from the reservoir scale 130.

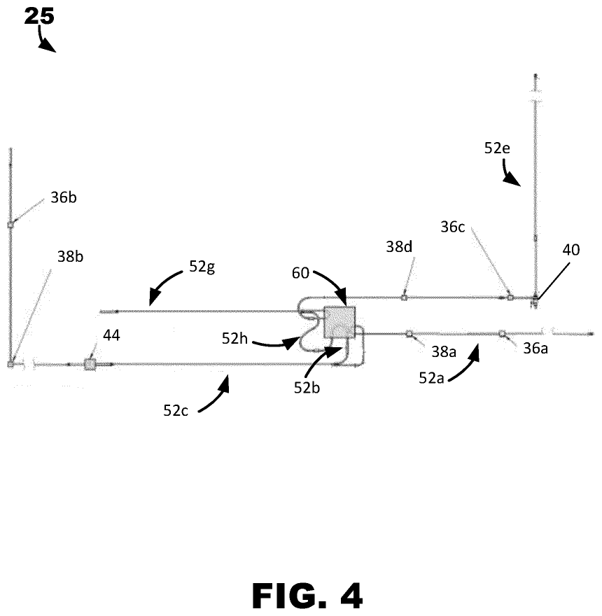

An illustrative fluid circuit and extracorporeal blood treatment apparatus arrangement that may be used by the extracorporeal treatment systems and apparatuses as described herein to perform an extracorporeal blood treatment on a patient using an organic filter operable to perform the functions of a human organ is depicted in FIG. 3. An illustrative treatment set is depicted in FIG. 4. Such illustrative fluid circuit and extracorporeal blood treatment apparatus arrangement and treatment sets may be used in conjunction with the extracorporeal treatment apparatus of FIG. 2 and the extracorporeal treatment system of FIG. 1. As shown in the exemplary figures, a blood treatment may be performed or executed by the exemplary extracorporeal blood treatment system, and thus, the treatment apparatus 24A (illustrated in FIG. 3) is configured to diagrammatically represent the exemplary physical extracorporeal fluid circuit for a blood treatment. The fluid lines corresponding to the exemplary blood treatment are depicted in FIGS. 3-5 as discussed herein. Additionally, the blood treatment may last for up to 72 hours at a time.

An exemplary treatment apparatus 24A is depicted in FIG. 3 that may be generally used during and/or for the execution of an extracorporeal blood treatment using an organic filter. The treatment apparatus 24A may include a treatment set 50 (which includes tubing such as tubing lines 52a-f as illustrated) which forms, or defines, a fluid circuit, where the treatment apparatus 24A is configured to diagrammatically represent the physical extracorporeal fluid circuit for an extracorporeal blood treatment performed by the exemplary extracorporeal blood treatment system using an organic filter. The treatment set 50 is indicated by the arrows which connect the various components. The treatment set 50 is operatively couplable to a patient 34 to move blood from the patient 34 to an organic filter 30 and to return the filtered blood to the patient 34. The treatment set 50 is operably couplable at least to the organic filter 30, a buffer reservoir 32, and the patient 34.

The treatment set 50 may further include one or more tubing lines, or segments, 52a-f (collectively referred to as 52) extending between and connecting various components of the exemplary fluid circuit. The various components of the exemplary fluid circuit include the organic filter 30, the buffer reservoir 32, the patient 34, one or more pressure sensors 36a-c (collectively referred to as 36), one or more sample ports 38a-d (collectively referred to as 38), a deaeration chamber 40, an air detector 42, a return tubing clamp 46, an oxygenator 44, a first low flow pump P1, a second low flow pump P2, and a high flow pump P3. The fluid flow direction through the treatment set 50 is depicted using the direction of the arrows. Each of the tubing lines 52, and each individual section of each of the tubing lines between adjacent components, and each of the components, may correspond to a fluid, flow or pump rate, pressure, and/or concentration of a fluid in a physical fluid circuit used in an exemplary extracorporeal blood treatment.

Additionally, the treatment set 50 is operably couplable to the patient 34, the organic filter 30, and the buffer reservoir 32. Such operable couplings may include, for example, intravenous access needles, catheters, or lines. The treatment set 50 is also operably couplable to the components of the treatment apparatus 24A discussed herein, and such operable couplings may include, for example, access ports or other mechanical couplings. The treatment apparatus 24A is sterile as it is configured to be used in medical settings.

The treatment set 50 and various components may provide the following fluid circuit path. For example, the treatment set 50 includes an access line 52a operatively couplable to the patient 34 and configured to receive fluid (e.g., whole blood) from the patient 34. Blood may be drawn from the patient 34 using the first low flow pump P1, and the blood may flow through an access pressure sensor 36a and past a first sample port 38a. Thus, the first low flow pump P1 can move blood through the treatment set from the patient 34 towards the organic filter 30. The access pressure sensor 36a may be configured to sense an access pressure of the blood within the treatment set 50 downstream from a patient access site (where the treatment set 50 is operably coupled to the patient 34). In one or more embodiments, the first sample port 38a is positioned downstream from the access pressure sensor 36a.

Each of the sample ports 38 may be configured to remove a fluid (e.g., blood) sample from the fluid in the treatment set 50. Samples can be used to test for various variables and substances (e.g., ammonia, toxins, blood cell counts, etc.). For example, samples may be taken upstream and downstream of the organic filter 30 to test for ammonia in the blood, in order to determine if the organic filter 30 is successfully filtering ammonia out of the blood.

The treatment set 50 also includes a buffer reservoir line 52g operatively couplable to the buffer reservoir and configured to receive fluid (e.g., whole blood) from the buffer reservoir. Blood may be drawn from the buffer reservoir 32 using the high flow pump P3. Thus, the high flow pump P3 can move blood through the treatment set (including the buffer reservoir line 52g, a high flow pump inlet line 52h, and a high flow pump outlet line 52b) to the organic filter 30. The fluid pumped through the access line 52a and the high flow pump outlet line 52b, respectively using the high flow pump P3 and the first low flow pump P1, may be combined as depicted in FIG. 3. This combined flow may be described as an “organic filter inlet flow.” The treatment set 50 also includes a high flow pump inlet line 52h which operatively couples the buffer reservoir line 52g to the high flow pump P3.

The treatment set also includes an organic filter inlet line 52c operatively couplable to the access line 52a, the buffer reservoir line 52g (via the high flow pump inlet line 52h and the high flow pump outlet line 52b), and the organic filter 30, and configured to move blood from the access line 52a and the buffer reservoir line 52g (via the high flow pump inlet line 52h and the high flow pump outlet line 52b) to the organic filter 30. This combined organic filter inlet flow allows the patient 34 to experience lower flow rates of the blood being removed from the patient 34, which may be safer or more comfortable for the patient 34. The combined organic filter inlet flow is discussed further herein.

In alternative embodiments, unfiltered blood is drawn not from a patient's vasculature, but from a storage container (e.g., a bag of blood). In such alternative embodiments, for example, there is less danger of withdrawing blood from the storage container too quickly (compared to withdrawing blood from a patient), and thus there may be no need for a buffer reservoir. Further, for example, the first low flow pump may then be configured to pump at higher speeds, and a high flow pump may not be necessary. Additionally, contrary to the nomenclature, the first and second low flow pumps may be configured to pump fluid at higher flow rates than the high flow pump. The nomenclature is not meant to be limiting in terms of absolute flow rate, but instead to differentiate between the pumps.

The organic filter inlet flow may move through an oxygenator 44, past a second sample port 38b, and through an organic filter pressure sensor 36b, before entering the organic filter 30. The oxygenator 44 may be configured to oxygenate the fluid within the treatment set 50. The oxygenator 44 may be positioned downstream from the high flow pump P3 and the first low flow pump P1, and may be positioned upstream from the organic filter 30. The organic filter pressure sensor 36b may be configured to sense organic filter pressure of the blood within the treatment set 50 upstream from the organic filter 30. The second sample port 38b may be positioned upstream from the organic filter pressure sensor 36b.

The organic filter 30 may be a liver organ, such as a human liver or a porcine liver that has been decellularized and then recellularized with human cells as described herein. The organic filter 30 may be a kidney organ, such as a human kidney or a porcine kidney, etc. The organic filter 30 may be a lung or lungs, such as a human lung(s) or a porcine lung(s), etc. The organic filter 30 may use a certain volume of blood flowing through it at a certain flow rate to function properly. For example, a human liver may hold about one pint of blood at any given time, and may filter blood at a rate of 1.7 liters/minute. Without a proper volume and flow rate of blood in and through the organic filter 30, the organic filter 30 may not function properly or may become damaged. To prevent this, the extracorporeal blood treatment system 10 may be configured to control the amount and speed of blood flowing in and through the organic filter 30 during a treatment process.

Once the blood has moved through the organic filter 30, it exits the organic filter 30 into an organic filter outlet line 52d operatively couplable to the organic filter and the buffer reservoir. The organic filter outlet line 52d may be configured to move blood from the organic filter 30 to the buffer reservoir 32. The fluid flow out of the organic filter 30 may move past a third sample port 38c and enter the buffer reservoir 32.

The organic filter 30 may be located in a container or basin (e.g., an airtight container or basin). The organic filter 30 may weep or leak some blood or other fluids during operation, and thus, the container may include a drain such that the blood or fluids wept, or leaked blood or fluids may be recaptured. Thus, the treatment set 50 may include a runoff line 52f operatively couplable to the drain of the container and the buffer reservoir 32 so as to move fluid runoff from the organic filter to the buffer reservoir 32. In other words, the runoff line 52f drains any fluid runoff from the organic filter 30 into the buffer reservoir 32. This drainage may be gravity driven or may be pump driven in alternative embodiments. Fluid runoff may include any fluid which the organic liver excretes during the treatment process that does not flow through the organic filter outlet line 52d.

The treatment set 50 also includes a return line 52e operatively couplable to the buffer reservoir 32 (via the buffer reservoir line 52g) and the patient 34 and configured to move blood from the buffer reservoir 32 to the patient 34. Thus, the buffer reservoir line 52g may be operatively coupled to the return line 52e, as illustrated in FIG. 3. The fluid is pumped from the buffer reservoir 32 using the second low flow pump P2, and the fluid is moved past a fourth sample port 38d, a return pressure sensor 36c, the deaeration chamber 40, the air detector 42, and the return tubing clamp 46 before returning to the patient 34. Thus, the second low flow pump P2 can move blood through the treatment set from the organic filter 30 to the patient 34. The second low flow pump P2 may be configured to return blood from the organic filter 30 to the patient 34 at a second low flow rate.

The return pressure sensor 36c may be configured to sense a return pressure of the blood within the treatment set 50 proximate a patient return site (where the treatment set 50 is operably coupled to the patient 34). The fourth sample port 38d may be positioned upstream from the return pressure sensor 36c. The deaeration chamber 40 may be positioned downstream from the return pressure sensor. The air detector 42 may be positioned downstream from the deaeration chamber 40. The return tubing clamp 46 may be positioned downstream from the air detector 42. Other configurations and positions of the various components may be configured to perform the same or similar functions as those discussed herein.

The computing apparatus 12 may be configured to stop the first low flow pump P1 and the second low flow pump P2, and close the return tubing clamp 46, when the air detector 42 detects air within the fluid in the treatment set 50. The computing apparatus 12 may be further configured to stop the high flow pump P3 if desired, or may be configured to continue using the high flow pump P3, for example, to prevent clotting. It can be dangerous and life-threatening to introduce air into the venous system of the patient 34, and this safety measure may prevent such dangers. In alternative embodiments, the air detector 42 may detect air bubbles of a minimum size to prevent such dangers.

As illustrated in FIG. 3, the treatment apparatus 24A further comprises the buffer reservoir 32 coupled to the treatment set 50. For example, as shown in FIG. 3, one of the fluid connections to the buffer reservoir 32 is shown extending to the buffer reservoir 32 from the organic filter 30, which may indicate that the buffer reservoir 32 stores and collects fluid (e.g., fluid from the patient, etc.). Further, in other areas of the treatment apparatus 24A, the fluid connection may be shown extending from the buffer reservoir 32 to the high flow pump P3 and the second low flow pump P2, which may indicate that the reservoir stores and supplies fluid to be used in the blood treatment process. The buffer reservoir 32 may correspond to a physical reservoir such as, e.g., one of the reservoirs 132 of the extracorporeal blood treatment system 100 described herein with respect to FIG. 2. The reservoirs 132 may be fluid bags as shown, or may be, e.g., cylinders, jugs, syringes, flasks, etc.

In such a configuration as illustrated, the computing apparatus 12 may control the treatment apparatus 24A so that, prior to performing the extracorporeal blood treatment, buffer reservoir 34 is partially or completely filled to hold a buffer volume of fluid (e.g., blood). For example, the buffer reservoir 32 may be filled with 1 liter of blood from the patient 34 and/or a non-patient blood supply, to be used in the extracorporeal blood treatment. Further, for example, filtered patient blood may be used to fill the buffer reservoir 32, or filtered non-patient blood may be used to fill the buffer reservoir 32.

The buffer reservoir 32 may be configured to contain, or hold, between 0 liters of blood and 5 liters of blood. During the illustrative treatment, the buffer reservoir 32 may be filled to include 1 liter of blood. In other embodiments, the buffer reservoir 32, during the illustrative treatment, may be filled to include greater than or equal to 0.25 liters of blood, greater than or equal to 0.5 liters of blood, greater than or equal to 0.75 liters of blood, greater than or equal to 1 liter of blood, greater than or equal to 1.25 liters of blood, greater than or equal to 1.5 liters of blood, etc. and/or less than or equal to 5 liters of blood, less than or equal to 3 liters of blood, less than or equal to 2 liters of blood, less than or equal to 1.5 liters of blood, less than or equal to 1.175 liters of blood, less than or equal to 0.95 liters of blood, etc.

Blood may be drawn from the patient 34 at a slower rate than the organic filter 30 requires, in order to provide safe and comfortable treatment to the patient. The computing apparatus 12 may further control the treatment apparatus 24A so that the high flow pump P3 is utilized to move blood from the buffer reservoir 32 to the organic filter 30 at a high flow rate, and so that the first low flow pump P1 is utilized to move blood from the patient 34 to the organic filter 30 at a first low flow rate. As illustrated in FIG. 3, the fluid circuit may promote blood flow from the patient 34 using the first low flow pump P1 and the fluid circuit may promote blood flow from the buffer reservoir 32 using high flow pump P3, and combine these flows towards the organic filter 30. Thus, the high flow rate and the first low flow rate are joined into a combined flow rate (as controlled by the computing apparatus 12), and the combined flow rate is configured to move a sufficient volume blood to the organic filter 30 at a sufficient flow rate to facilitate functionality of the organic filter.

In one or more embodiments, the first low flow pump P1 may be configured to move blood from the patient 34 to the organic filter 30 at a first low flow rate and the second low flow pump P2 may be configured to return blood from the organic filter 30 to the patient 34 at a second low flow rate. Further, the first low flow rate may be equal to the second low flow rate. This may assist in ensuring that the fluid being drawn from the patient 34 is replaced by an equal amount of fluid that returns to the patient 34.

The first low flow rate may be between about 50 milliliters per minute and about 170 milliliters per minute. In at least one embodiment, the first low flow rate is 166 milliliters per minute. In at least one embodiment, the first low flow rate may be greater than or equal to 50 milliliters per minute, greater than or equal to 100 milliliters per minute, greater than or equal to 125 milliliters per minute, greater than or equal to 150 milliliters per minute, and/or less than or equal to 175 milliliters per minute, less than or equal to 160 milliliters per minute, less than or equal to 140 milliliters per minute, less than or equal to 110 milliliters per minute, less than or equal to 85 milliliters per minute, etc.

The high flow rate may be between about 200 milliliters per minute and about 500 milliliters per minute. In at least one embodiment, the high flow rate is 440 milliliters per minute. In at least one embodiment, the high flow rate is 184 milliliters per minute. In at least one embodiment, the high flow rate may be greater than or equal to 200 milliliters per minute, greater than or equal to 250 milliliters per minute, greater than or equal to 300 milliliters per minute, greater than or equal to 400 milliliters per minute, greater than or equal to 450 milliliters per minute, etc. and/or less than or equal to 475 milliliters per minute, less than or equal to 425 milliliters per minute, less than or equal to 375 milliliters per minute, less than or equal to 325 milliliters per minute, etc.

The second low flow rate may be between about 50 milliliters per minute and about 170 milliliters per minute. In at least one embodiment, the second low flow rate is 166 milliliters per minute. In at least one embodiment, the second low flow rate may be greater than or equal to 50 milliliters per minute, greater than or equal to 100 milliliters per minute, greater than or equal to 125 milliliters per minute, greater than or equal to 150 milliliters per minute, and/or less than or equal to 175 milliliters per minute, less than or equal to 160 milliliters per minute, less than or equal to 140 milliliters per minute, less than or equal to 110 milliliters per minute, less than or equal to 85 milliliters per minute, etc.

The computing apparatus 12 (illustrated in FIG. 1) may be operatively coupled to the extracorporeal blood treatment apparatus 24, 24A (illustrated in FIGS. 1-3). The computing apparatus 12 may be configured to perform an extracorporeal blood treatment on the patient 34 using the extracorporeal blood treatment apparatus 24 and the organic filter 30. As described herein, the computing apparatus 12 may direct the flow of fluid through the treatment set 50 using the pumps (P1-P3) to remove blood from the patient 34, move the blood through the organic filter 30, and return the blood to the patient 34.

The computing apparatus 12 may be further configured to utilize the first low flow pump Pl to draw blood from the patient 34 towards the organic filter 30 at a first low flow rate. The computing apparatus 12 may be further configured to utilize the second low flow pump P2 to return blood from the organic filter 30 to the patient 34 at a second low flow rate. The computing apparatus 12 may be further configured to utilize the high low flow pump P3 to draw blood from the buffer reservoir 32 towards the organic filter 30 at a high flow rate.

The computing apparatus 12 may be further configured to control the high flow rate, the first low flow rate, and the second low flow rate based on at least one of the access pressure, the organic filter pressure, and the return pressure. The computing apparatus 12 may be further configured to use at least one of the high flow pump P3, the first low flow pump P1, and the second low flow pump P2 to maintain the access pressure within access pressure limits, to maintain an organic pressure within organic filter pressure limits, and/or to maintain a return pressure within return pressure limits. In one or more embodiments, the organic filter pressure limit is greater than the return and access pressure limits. In one or more embodiments, the organic filter pressure limit is less than the return and access pressure limits. If any respective pressure limit is reached, the pumps are controlled to stop, and no fluid is moved through the treatment apparatus 24A.

The access pressure limit may be between about −250 mmHg and 400 mmHg. In at least one embodiment, the access pressure limit is −200 mmHg. In at least one embodiment, the access pressure limit is greater than or equal to −100 mmHg, greater than or equal to 0 mmHg, greater than or equal to 50 mmHg, greater than or equal to 25 mmHg, greater than or equal to 75 mmHg, greater than or equal to 100 mmHg, greater than or equal to 150 mmHg, greater than or equal to 200 mmHg, greater than or equal to 250 mmHg, greater than or equal to 300 mmHg, greater than or equal to 400 mmHg, and/or less than or equal to 375 mmHg, less than or equal to 275 mmHg, less than or equal to 175 mmHg, less than or equal to 90 mmHg, less than or equal to 45 mmHg, less than or equal −80 mmHg, etc.

The organic filter pressure limit may be between about 0 mmHg and 450 mmHg. In at least one embodiment, the organic filter pressure limit is 55 mmHg. In at least one embodiment, the organic filter pressure limit is greater than or equal to 0 mmHg, greater than or equal to 50 mmHg, greater than or equal to 25 mmHg, greater than or equal to 75 mmHg, greater than or equal to 100 mmHg, greater than or equal to 150 mmHg, greater than or equal to 200 mmHg, greater than or equal to 250 mmHg, greater than or equal to 300 mmHg, greater than or equal to 400 mmHg, greater than or equal to 450 mmHg, and/or less than or equal to 475 mmHg, less than or equal to 375 mmHg, less than or equal to 275 mmHg, less than or equal to 175 mmHg, less than or equal to 90 mmHg, less than or equal to 45 mmHg, etc.

The return pressure limit may be between about 0 mmHg and 300 mmHg. In at least one embodiment, the return pressure limit is 200 mmHg. In at least one embodiment, the return pressure limit is greater than or equal to 0 mmHg, greater than or equal to 50 mmHg, greater than or equal to 25 mmHg, greater than or equal to 75 mmHg, greater than or equal to 100 mmHg, greater than or equal to 150 mmHg, greater than or equal to 200 mmHg, greater than or equal to 250 mmHg, greater than or equal to 300 mmHg, and/or less than or equal to 375 mmHg, less than or equal to 275 mmHg, less than or equal to 175 mmHg, less than or equal to 90 mmHg, less than or equal to 45 mmHg, etc.

The treatment apparatus 24, 24A may further include a scale (e.g., 130 illustrated in FIG. 2) to weigh the buffer reservoir 32. The computing apparatus 12 may be further configured to use at least one of the high flow pump P3, the first low flow pump P1, and the second low flow pump P2 to maintain a weight of the buffer reservoir 32. In alternative embodiments, the computing apparatus 12 may use the pumps P1-P3 to maintain the weight of the buffer reservoir 32 within a range. Such range may be based on the initial weight of the buffer reservoir 32, and may be based on set values or a percentage of the initial weight of the buffer reservoir 32. If the weight of the buffer reservoir 32 is not maintained, the pumps may stop, and no fluid may be moved through the treatment apparatus 24A.

The flow rates of the pumps P1-P3 may change depending on the treatment and/or phase within the treatment and may be changed or modified by an operator. An operator, or user, may use input apparatus 20 of the exemplary extracorporeal blood treatment system 10 described herein with reference to FIG. 1 to select one or more portions such as, e.g., regions, areas, elements, items, icons, buttons, etc. of the graphical user interface 200. For example, the input apparatus 20 may be a touch screen that corresponds to the graphical user interface 200. As used herein, when an operator “selects” a portion of the graphical user interface, it is to be understood that selecting the portion may be conducted in many different ways using many different types of input apparatus. For example, when the input apparatus is a touch screen, an operator may select a portion by “touching” the portion with their finger or using a pointing device such as a stylus.

When an operator adjusts a flow rate of a particular pump, one or more adjustment notifications may be depicted, or displayed. In one or more embodiments, if one pump flow rate may be adjusted in order to maintain or obtain a desired variable (e.g., flow rate, pressure, weight), then the exemplary systems may provide automatic adjustment of the flow rate of another one of the pumps P1-P2. In other words, one flow rate may be dependent on another flow rate. Further, in one or more embodiments, one or more flow rates of one of the pumps P1-P3 may automatically adjust without user intervention based on the monitored variables (e.g., pressure, weight, etc.).

Further exemplary treatment apparatuses, or exemplary treatment sets 25, are depicted in FIG. 4. Such exemplary treatment apparatuses or sets 25 may be used in conjunction with the extracorporeal treatment apparatus of FIG. 2 or the extracorporeal treatment system of FIG. 1. Similar to the treatment apparatus 24A as described in FIG. 3, the treatment set 25 includes the access line 52a operatively couplable to the patient (not shown) and configured to receive blood from the patient. The treatment set 25 includes the buffer reservoir line 52g operatively couplable to the buffer reservoir (not shown) and configured to receive blood from the buffer reservoir. The treatment set 25 includes the high flow pump inlet line 52h operatively couplable to the buffer reservoir line 52g and the high flow pump P3. The treatment set 25 includes the high flow pump outlet line 52b operatively couplable to the high flow pump P3 and the access line 52a. The treatment set 25 includes the organic filter inlet line 52c operatively couplable to the access line 52a, the buffer reservoir line 52g (via the high flow pump inlet line 52h and the high flow pump outlet line 52b), and the organic filter (not shown), and configured to move blood from the access line and the buffer reservoir line to the organic filter. The treatment set includes the organic filter outlet line 52d operatively couplable to the organic filter and the buffer reservoir and configured to move blood from the organic filter to the buffer reservoir. The treatment set includes the return line 52e operatively couplable to the buffer reservoir (via the buffer reservoir line 52g) and the patient and configured to move blood from the buffer reservoir to the patient. The treatment set includes other components as illustrated and as discussed herein with respect to FIG. 3. For example, the cassette 60 may interface with pumps P1-P3. The treatment set 25 may have optimized dimensions for the specific blood treatment being performed.

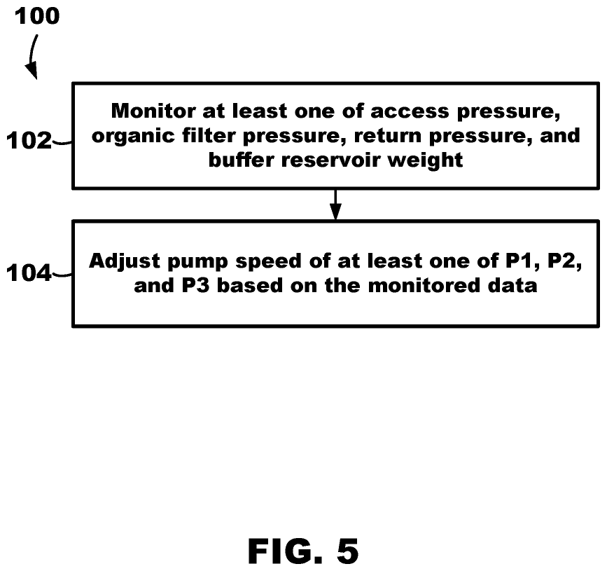

An illustrative method of performing an extracorporeal blood treatment using the illustrative apparatuses and/or systems is depicted in FIG. 5. The method 100 includes monitoring at least one of the access pressure, the organic filter pressure, the return pressure, and the buffer reservoir weight 102. Monitoring of the access pressure may be performed using the access pressure sensor 36a. Monitoring of the organic filter pressure may be performed using the organic filter pressure sensor 36b. Monitoring of the return pressure may be performed using the return pressure sensor 36c. Monitoring of the buffer reservoir weight may be performed using the reservoir scale 130. The method 100 may further include adjusting the pump speed of at least one of the first low flow pump P1, the second low flow pump P2, and the high flow pump P3 based on the monitored data 104. Adjusting the pump speed may be done using the computing apparatus 12 as described herein.

All patents, patent documents, and references cited herein are incorporated in their entirety as if each were incorporated separately. This disclosure has been provided with reference to illustrative embodiments and is not meant to be construed in a limiting sense. As described previously, one skilled in the art will recognize that other various illustrative applications may use the techniques as described herein to take advantage of the beneficial characteristics of the systems and methods described herein. Various modifications of the illustrative embodiments, as well as additional embodiments of the disclosure, will be apparent upon reference to this description.

1. An apparatus to perform an extracorporeal blood treatment using an organ operable to perform functions of a human organ, the apparatus comprising:
a treatment set comprising tubing operatively couplable to a patient, wherein the treatment set is configured to move blood from the patient to the organ and to return filtered blood from the organ to the patient,
a high flow pump operatively couplable to the treatment set to move blood through the treatment set towards the organ;
a first low flow pump operatively couplable to the treatment set to move blood through the treatment set from the patient towards the organ;
a second low flow pump operatively couplable to the treatment set to move blood through the treatment set from the organ towards the patient; and
a computing apparatus operatively coupled to the pumps and configured to perform an extracorporeal blood treatment by controlling the pumps.

2. The apparatus of claim 1, further comprising a buffer reservoir coupled to the treatment set, wherein the computing apparatus is further configured to:
prior to performing the extracorporeal blood treatment, fill the buffer reservoir to hold a buffer volume of blood;
control the high flow pump to move blood from the buffer reservoir to the organ at a high flow rate, and
control the first low flow pump to move blood from the patient to the organ at a first low flow rate.

3. The apparatus of claim 2, wherein the computing apparatus is further configured to control the high flow rate and the first low flow rate into a combined flow rate, and wherein the combined flow rate is configured to move a sufficient volume of blood to the organ at a sufficient flow rate to facilitate functionality of the organ.

4. The apparatus of claim 1, wherein the computing apparatus is further configured to control the second low flow pump to return blood from the organ to the patient at a second low flow rate.

5. The apparatus of claim 1, wherein the first low flow pump is configured to move blood from the patient to the organ at a first low flow rate and the second low flow pump is configured to return blood from the organ to the patient at a second low flow rate, wherein the first low flow rate is equal to the second low flow rate.

6. The apparatus of claim 1, wherein the treatment set comprises:
an access line operatively couplable to the patient and configured to receive blood from the patient;
a buffer reservoir line operatively couplable to the buffer reservoir and configured to receive blood from the buffer reservoir;
an organic filter inlet line operatively couplable to the access line, the buffer reservoir line, and the organ, and configured to move blood from the access line and the buffer reservoir line to the organ;
an organic filter outlet line operatively couplable to the organ and the buffer reservoir and configured to move blood from the organ to the buffer reservoir; and
a return line operatively couplable to the buffer reservoir and the patient and configured to move blood from the buffer reservoir to the patient.

7. The apparatus of claim 6, wherein the treatment set further comprises:
a runoff line operatively couplable to the buffer reservoir and configured to move fluid runoff from the organ to the buffer reservoir.

8. The apparatus of claim 6, wherein the treatment set further comprises:
an access pressure sensor configured to sense an access pressure of the blood within the treatment set downstream from a patient access site;
an organic filter pressure sensor configured to sense an organic filter pressure of the blood within the treatment set upstream from the organ; and
a return pressure sensor configured to sense a return pressure of the blood within the treatment set proximate a patient return site.

9. The apparatus of claim 8, wherein the computing apparatus is operatively coupled to the pressure sensors and is further configured to control at least one of the high flow pump, the first low flow pump, and the second low flow pump to maintain the access pressure within access pressure limits, to maintain an organic pressure within organic filter pressure limits, and to maintain a return pressure within return pressure limits.

10. The apparatus of claim 9, wherein the access pressure limits are greater than or equal to −250 mmHg and are less than or equal to 150 mmHg.

11. The apparatus of claim 9, wherein the return pressure limits are greater than or equal to 50 mmHg and are less than or equal to 300 mmHg.

12. The apparatus of claim 2, wherein the extracorporeal blood treatment apparatus further comprises a scale configured to weigh the buffer reservoir, and wherein the computing apparatus is further configured to control at least one of the high flow pump, the first low flow pump, and the second low flow pump to maintain a buffer weight of the buffer reservoir.

13. The apparatus of claim 1, wherein the treatment set further comprises a deaeration chamber, an air detector, and a return tubing clamp,
wherein the deaeration chamber is positioned downstream from a return pressure sensor, and wherein the air detector is positioned downstream from the deaeration chamber, and wherein the return tubing clamp is positioned downstream from the air detector, and
wherein the computing apparatus is configured to stop the first low flow pump and the second low flow pump, and close the return tubing clamp, when the air detector detects air.

14. The apparatus of claim 1, wherein the treatment set further comprises an oxygenator configured to oxygenate the blood,
wherein the oxygenator is positioned downstream from the high flow pump and the first low flow pump and upstream from the organ.

15. (canceled)

16. The apparatus of 15 claim 14, wherein the organ is operable to perform functions of at least one of a human liver organ, a human kidney organ, and a human lung organ.

17-30. (canceled)

31. An extracorporeal blood treatment apparatus comprising:
a treatment set comprising tubing operatively couplable to a patient, wherein the treatment set is configured to move blood from the patient to an organ operable to perform functions of a human organ, and to return filtered blood to the patient, the treatment set comprising:
an access line operatively couplable to a patient and configured to receive blood from the patient;
an organic filter inlet line operatively couplable to the access line and to the organ, the organic filter inlet line being configured to move blood from the access line to the organ;
a return line operatively couplable to the patient and configured to move blood back to the patient; and
a high flow pump outlet line operatively couplable to at least one of the access line and the organic filter inlet line and configured to move blood from the organ to at least one of the access line and the organic filter inlet line;
a first low flow pump operatively couplable to the access line and configured to move blood through the treatment set from the patient towards the organ;
a second low flow pump operatively couplable to the return line and configured to move blood through the treatment set from the organ towards the patient;
a high flow pump operatively couplable to the high flow pump outlet line and configured to move blood from the organ to at least one of the access line and the organic filter inlet line and further move blood to the organ;
a computing apparatus operatively couplable to the pumps and configured to control the first low flow pump to move blood through the treatment set from the patient towards the organ, to control the second low flow pump to move blood through the treatment set from the organ towards the patient, and to control the high flow pump to move blood from the organ to either the access line or the organic filter inlet line, and further move blood to the organ.

32. The apparatus of claim 31, further comprising:
a buffer reservoir;
an organic filter outlet line operatively couplable to the organ and the buffer reservoir and configured to move blood from the organ to the buffer reservoir; and
a buffer reservoir line operatively couplable to the buffer reservoir and to the high flow pump outlet line and configured to move blood from the buffer reservoir to the high flow pump,
wherein the computing apparatus is configured to control the high flow pump to move blood from the buffer reservoir to at least one of the access line and the organic filter inlet line, and further move the blood to the organ.

33-34. (canceled)

35. The apparatus of claim 32, further including a runoff line operatively couplable to a container housing the organ and to the buffer reservoir and configured to drain fluid runoff from the organ to the buffer reservoir.

36. (canceled)

37. The apparatus of claim 32, wherein the computing apparatus is further configured to:
prior to performing the extracorporeal blood treatment, fill the buffer reservoir to hold a buffer volume of blood;
control the high flow pump to move blood from the buffer reservoir to the organ at a high flow rate;
control the first low flow pump to move blood from the patient to the organ at a first low flow rate; and
control the high flow rate and the first low flow rate into a combined flow rate, and wherein the combined flow rate is configured to move a sufficient volume of blood to the organ at a sufficient flow rate to facilitate functionality of the organ.

38. The apparatus of claim 16, wherein the treatment set further comprises:
an access pressure sensor configured to sense an access pressure of the blood within the treatment set downstream from a patient access site;
an organic filter pressure sensor configured to sense an organic filter pressure of the blood within the treatment set upstream from the organ; and
a return pressure sensor configured to sense a return pressure of the blood within the treatment set proximate a patient return site,
wherein the computing apparatus is operatively coupled to the pressure sensors and is further configured to control at least one of the high flow pump, the first low flow pump, and the second low flow pump to maintain the access pressure within access pressure limits, to maintain an organic pressure within organic filter pressure limits, and to maintain a return pressure within return pressure limits.
